SEQUENCE LISTING (2 sequences)

SEQ ID NO: 1 - DNA, 20 bp, Artificial Sequence
  Description of Artificial Sequence primer
ttacaagcctgatgaaggga

SEQ ID NO: 2 - DNA, 28 bp, Artificial Sequence
  Description of Artificial Sequence primer
ccatgaatagcactgggagcattgaagc

(19) United States
(12) Patent Application Publication
(10) Pub. No.: US 2004/0254127 A1
(43) Pub. Date: Dec. 16, 2004

(54) Method for determining predisposition to a venous thromboembolic disease

(76) Inventors: Joseph Emmerich, Paris (FR); Jean Noel Fiessinger, Paris (FR); Martine Aiach, Sevres (FR)

Correspondence Address: STITES & HARBISON PLLC, 1199 NORTH FAIRFAX STREET, SUITE 900, ALEXANDRIA, VA, 22314, US

(21) Appl. No.: 10/830,094

(22) Filed: Apr. 23, 2004

Related U.S. Application Data
(62) Division of application No. 09/937,035, filed on Nov. 19, 2001, now abandoned.
Application No. 09/937,035 is a 371 of international application No. PCT/FR00/00725, filed on Mar. 22, 2000.

(30) Foreign Application Priority Data
Mar. 23, 1999 (FR) 99 03613

(51) Int. Cl. A61K 31/70; A61K 31/65; A61K 31/7048; A61K 31/496; A61K 31/4741

(52) U.S. Cl. 514/29; 514/152; 514/291; 514/252.13

(57) ABSTRACT

The invention concerns a method for detecting in vitro predisposition to a venous thromboembolic disease in a subject, which consists in determining whether the subject has been infected by a genus Chlamydia bacterium, in particular Chlamydia pneumoniae. The invention also concerns the use of active agents on genus Chlamydia bacteria for preventing and/or treating a venous thromboembolic disease.

The present invention is based on the demonstration of a close link between infection with a bacterium of the Chlamydia genus and venous thromboembolic disease.

The invention more particularly relates to a method for determining, in vitro, predisposition to a venous thromboembolic disease in an individual, in which it is determined whether the individual has been infected with a bacterium of the Chlamydia genus, and to the use of agents active on bacteria of the Chlamydia genus, for preventing and/or treating a venous thromboembolic disease.

Venous thromboembolic disease is a multifactor disorder with both genetic and acquired risk factors (Rosendaal et al., 1997). Conventional risk factors for venous thrombosis are associated with modification of the vessels or stasis (slowing down, or even stopping of venous blood circulation), due in particular to a trauma, a surgical operation or immobilization. Overall, genetic predisposition to venous thrombosis explains only 40% of cases. Deficiencies in antithrombin, in protein C and in protein S explain in particular 5 to 10% of cases of venous thromboembolic diseases. After having taken into account conventional acquired risk factors and genetic predispositions, at least a third of venous thrombotic episodes remain unexplained.

Currently, the most commonly used treatment in cases of venous thrombosis is treatment based on anticoagulants. However, such a treatment can be envisioned only for a limited period of time, because of dangerous side effects. Prolonged administration of anticoagulants in fact increases the risks of hemorrhages, in particular cerebral hemorrhages. Consequently, an alternative treatment is sought.

The link between infection with Chlamydia pneumoniae and atherosclerosis is well documented. The use of antibiotics in the treatment of cardiovascular diseases, such as myocardial infarction or coronary artery diseases, has also been envisioned (WO 90/00061, WO 98/17280, WO 98/06408, Gibbs et al., 1998). The cardiovascular diseases targeted are, however, diseases of the arterial system.

On the other hand, to date, no datum would suggest the possibility of a link between Chlamydia pneumoniae and the pathologies of the venous system. Although an article by Ong et al., published in 1996, reported the detection by PCR (polymerase chain reaction) of Chlamydia pneumoniae in an iliac vein on two control individuals a priori free of pathological vascular conditions, a subsequent article (Bartels et al., 1998) reported, on the contrary, the absence of this bacterium in the native saphenous veins of patients who had undergone a coronary bypass.

The authors of the present invention have discovered, surprisingly, a close link between infection with a bacterium of the Chlamydia genus and a venous thromboembolic disease.

More particularly, the authors of the invention have shown that high levels of anti-Chlamydia pneumoniae antibodies represent a risk factor for a venous thromboembolic disease.

Without in any way associating themselves with a precise mechanism of action, the authors are putting forward the hypothesis that chronic infection of the vein walls with Chlamydia pneumoniae may render the venous endothelial cells thrombogenic.

A subject of the present invention is therefore a method for determining, in vitro, predisposition to a venous thromboembolic disease in an individual, in which it is determined whether the individual has been infected with a bacterium of the Chlamydia genus, more particularly Chlamydia pneumoniae.

It is determined whether the individual has been infected with a bacterium of the Chlamydia genus by analyzing a biological sample. It may in particular be a sample of blood, of urine or of pleural liquid, a sample obtained by bronchoscopy or by bronchoalveolar lavage, or a sample obtained by biopsy, for example of the vascular endothelium. It is then possible to determine whether this sample contains anti-Chlamydia antibodies or whether it contains bacteria of the Chlamydia genus or fragments thereof. It is, for example, possible to search for the presence of a chlamydial component, such as liposaccharides or membrane-bound proteins, of substances produced by Chlamydia, such as exopolysaccharides, or of substances produced by the host cells via Chlamydia induction.

Preferably, it is determined whether the individual has been infected with a bacterium of the Chlamydia genus, by assaying the level of anti-Chlamydia antibodies in a biological sample from an individual to be tested, for Hi example a blood sample.

The anti-Chlamydia antibody titers obtained in the individuals to be tested are then compared with the antibody titers obtained in control individuals. The titer is defined by the maximum dilution of the biological sample for which the antibodies are still detected, and is expressed by the inverse of the dilution factor. An antibody titer of greater than 256 may be considered to represent a not insignificant risk factor.

A subject of the present invention is therefore, more particularly, a method for determining predisposition to a venous thromboembolic disease in an individual, comprising the steps consisting in:

i) assaying the level of anti-chlamydia antibodies in a biological sample from an individual to be tested;

ii) comparing this level of antibodies with the level of anti-chlamydia antibodies obtained in control individuals;

iii) identifying the individual tested as an individual exhibiting predisposition to a venous thromboembolic disease if the level of antibodies obtained in step i) is greater than the level of anti-chlamydia antibodies obtained in control individuals.

The present invention also relates to the use of at least one agent active against infection with a bacterium of the chlamydia genus, in particular Chlamydia pneumoniae, or of at least one agent effective against the inflammatory effects of infection with Chlamydia, for preventing and/or treating a venous thromboembolic disease.

Preferably, pharmaceutical compositions containing antibiotic agents may be used.

A subject of the invention is therefore more particularly the use of an antibiotic substance active on bacteria of the Chlamydia genus, in particular Chlamydia pneumoniae, for preparing a medicinal product intended to prevent and/or treat a venous thromboembolic disease.

Among the antibiotic substances or agents active on the bacterial genus Chlamydia, mention may in particular be made of macrolides (for example erythromycin or azithromycin), tetracyclines, fluoroquinolones and rifampicin.

According to another embodiment of the present invention, said agent active against infection with Chlamydia may be an immunogenic protein, or an immunogenic fragment of a protein, of Chlamydia, in particular of Chlamydia pneumoniae. These proteins, or these protein fragments, are characterized by their ability to induce cell-mediated or humoral immunity against infection with Chlamydia, in particular Chlamydia pneumoniae, by administration of the protein in combination with a suitable adjuvant. Preferably, use may be made of the major outer membrane protein or cell surface proteins from the bacterium, which may be randomly fragmented. The random fragments of a Chlamydia protein can be tested for their immunogenicity by those skilled in the art. Vaccines containing cellular antigens of Chlamydia [lacuna] fragments thereof may be obtained conventionally, for example by cell lysis or using standard purification or separation techniques.

A composition which can be used according to the invention may also comprise a Chlamydia bacterium which has been killed or inactivated by any conventional means, such as heat or irradiation.

A composition which can be used according to the present invention may also contain one or more nucleic acid sequences encoding a surface protein of Chlamydia or a fragment thereof. The nucleic acid used may be administered using an immunization vector or in naked form, i.e. free of any agent which facilitates the penetration of this nucleic acid into the cell.

A subject of the invention is also a method for preventing and/or treating venous thromboembolic disease, in which a prophylactically or therapeutically effective amount of an agent active on bacteria of the Chlamydia genus, in particular of an antibiotic agent, in combination with a pharmaceutically acceptable vehicle, is administered to a patient requiring such a treatment.

The prevention of recurrences subsequent to a first venous thrombosis is more particularly targeted.

The methods of an administration, the doses and the pharmaceutical forms of the pharmaceutical compositions which can be used according to the invention may be determined in the usual way by those skilled in the art, in particular according to the criteria generally taken into account in establishing a therapeutic treatment suitable for a patient, such as for example the age or body weight of the patient, the seriousness of his or her general condition, the tolerance to the treatment and the side effects noted, etc.

A pharmaceutical composition which can be used according to the invention may in particular be administered orally, parenterally, intravenously, intramuscularly, subcutaneously, percutaneously or intranasally.

When the pharmaceutical composition used is an antibiotic composition, the effective dose lies within the ranges conventionally used for any antibiotic Hi against Chlamydia bacteria. Said composition may in particular be advantageously administered in short cycles (4 to 10 days approximately), to be repeated, for example, every six months after the first venous thrombotic episode.

The link between infection with a Chlamydia pneumoniae bacterium and a venous thromboembolic disease is illustrated in the results presented hereinafter, which in no way limit the scope of the present invention.

The authors of the present invention have thus studied 176 patients with a diagnosed venous thromboembolic disease and 197 control individuals in good health, of various age and sex. The acquired risk factors for a venous thromboembolic disease and the common genetic predisposition factors (Arg 506 Gln mutation in factor V and G 20210 A mutation in factor II) were evaluated in all the individuals. The level of anti-C. pneumoniae IgG antibodies was determined by microimmuno-fluorescence. All the positive plasma samples (titer ≦128) were accurately quantified and tested for the presence of specific IgM antibodies.

Materials and Methods:

Individuals:

The patients less than 61 years old who had had at least one episode of deep vein thrombosis diagnosed objectively (compression ultrasonography or venography) and/or a pulmonary embolism (ventilation and perfusion pulmonary scintigraphy, conventional pulmonary angiography and a spiral scan) were selected from 205 patients (92 men and 113 women) with venous thromboembolic diseases. A complete clinical study was carried out on all the patients, emphasizing their personal and family histories regarding thromboembolic disease, and the acquired risk factors (surgery or trauma within the last three months, immobilization for longer than 72 hours, pregnancy, treatment with estrogens, varicose veins and cancer). Blood was taken from 176 patients (86%); in 87 cases, samples were obtained less than three months after an acute venous thromboembolic disease had been triggered (median 1 day; IQR deviation 0-16), whereas the remaining 89 samples were obtained more than three months after triggering (median 12 months, IQR deviation 6.5-36). The 197 healthy control individuals of varied age and sex were selected as having no history of venous thromboembolic disease, of myocardial infarction or of peripheral vascular disease.

Assay:

The venous blood was collected in 0.129 M trisodium citrate (1:10) and two steps of centrifugation at 2000 g for 15 minutes were carried out in order to obtain a platelet-depleted plasma. The plasma was frozen and stored in aliquot fraction form at −40° C. until use. The DNA was extracted from the leukocytes using standard methods and stored at 4° C.

For the serological tests, each case and each control sample were labeled with a random number and analyzed blind. The serological status in terms of C. pneumoniae was determined by a microimmunofluorescence (MIF) assay using the “SeroFIA IgG Chlamydia” kit (Savyon Diagnostics Ltd., Ashdod, Israel). Briefly, elementary bodies of purified C. pneumoniae (IOL 207 strain) were used to detect the IgG antibodies. All the plasma samples were initially screened at a dilution of 1:128 and were considered to be positive above this dilution. The positive plasmas were then tested at dilutions of 1:256, 1:512 and 1:1024. The specific IgG titers were given as the inverse of the final positive dilution. Samples positive for anti-C. pneumoniae IgGs were then tested for the presence of anti-C. pneumoniae IgMs using the MIF assay with the “SeroFIA IgM Chlamydia” kit (Savyon Diagnostics Ltd), at a dilution of 1:20 as recommended by the manufacturer.

A study was, moreover, carried out to determine whether the DNA of the control individuals and of the patients exhibited the Arg 506 Gln mutation in factor V, after PCR amplification of exon 10 of factor V and digestion with restriction enzymes. The transition G 20210 A of the prothrombin gene was identified after amplification using two primers: (5′-TTACAAGCCTGATGAAGGGA-3′ and 5′-CCATGAATAGCACTGGGAGCATTGAAGC-3′). The second primer was , constructed such that a nucleotide substitution (C to A) at position 20210 creates a new restriction site for Hind III when the transition of G to A at position 20210 is present.

Statistical Analysis:

The data are analyzed using the SAS statistical program (Institute Inc., Cary, N.C.). The clinical characteristics of the entire population of patients and of the subgroup of cases with blood samples taken less than three months after the episode of thrombosis were compared to those of the control individuals using a Chi2 test with one degree of freedom. Age was tested using analysis of variance (ANOVA).

The odds ratios associated with seropositivity for C. pneumoniae were calculated by comparing the individuals having titers of 256 or more having individuals with titers of less than 256. The heterogeneity of the odds ratios in terms of age and sex was tested by entering the interaction variables (one degree of freedom) into logistic regressions. The odds ratios for a venous thromboembolic disease and the 95% confidence interval (CI) associated with each anti-C. pneumoniae IgG titer, coded as binary variables, were then calculated with reference to seronegativity using a logistic regression procedure (SAS-PROC LOGIST). The same analysis was applied to the subgroup of cases with blood samples taken less than three months after the venous thromboembolic disease had been triggered. In the entire population tested, the levels of seropositivity for C. pneumoniae were compared in terms of first thrombosis against recurrent thrombosis, in terms of presence of associated risk factor against absence of associated risk factor, and in terms of first thrombosis at under 40 years of age (median age of the population) against first thrombosis at over 40 years of age, using a Chi2 test with one degree of freedom. The differences with values p less than 0.05 were considered to be significant.

Results:

The cases and controls do not differ in terms of age ratios or sex ratios (cf. table 1). Venous thromboembolic disease was considered to be idiopathic in patients who had not been taking any oral contraception, who had not recently had surgery (less than one month), who had not suffered any trauma, who had not been pregnant or given birth, who had not been immobilized or who had not suffered from cancer. Half of the patients were recruited within three months after the acute episode of venous thrombosis. The characteristics of this subgroup were not statistically different from those of the entire population of cases. The prevalence of the Arg 506 Gln mutation of factor V and the G 20210 A mutation of factor II was within the range expected in Caucasians (21.9% in the patients and 5.1% in the controls, p<0.0001; and 10.2% and 4.1%, p=0.02, respectively).

The C. pneumoniae-specific IgG titers tend to be higher in the patients than in the controls (cf. table 2). Significantly, it is observed that more patients than controls have C. pneumoniae IgG titers of 256 or more (54% and 15.9% respectively, p<0.0001). The odds ratio for the venous thromboembolic diseases associated with IgG titers of 256 or more was 6.2 (95% confidence interval (CI), 3.8-10.1). In the subgroup of patients with blood samples taken less than three months after the thrombotic episode, the crude ratio (odds ratio) among the individuals with IgG titers >256 was 5.4 (95% CI, 3.1-9.6). In addition, the odds ratio for a venous thromboembolic disease increases with the IgG titer: for titers of 256, 512 and 1024, the crude odds ratios were 2.1 (95% CI, 1.0-4.2), 4.3 (2.1-8.9) and 32.4 (4.2-248.3), respectively (cf. table 3). A greater proportion of seropositive controls had a low IgG titer of 128 compared to the controls (54.9% and 21%, respectively). Similar odds ratios were obtained in the subgroup of cases tested within three months after the thrombotic episode.

The odds ratios were not significantly different according to age or sex. After exclusion of the individuals bearing the Arg 506 Gln mutation of factor V and the G 20210 A mutation of factor II, the odds ratios for the venous thromboembolic disease associated with a titer greater than 256 was 7.7 (95% CI, 4.5-13.2). The characteristics of the venous thromboembolic disease (age at the time of the first episode; recurrent or spontaneous nature) did not differ as a function of the serological status for C. pneumoniae.

In order to distinguish an acute infection from a chronic infection, the authors of the invention also evaluated the circulating anti-C. pneumoniae IgM antibodies in the 95 cases and 31 controls who had IgG titers greater than 256. Only one individual from the group of patients suffering from venous thromboembolic diseases was positive for IgMs.

Conclusion:

The results above clearly show a link between the serological status for C. pneumoniae and venous thrombosis. The odds ratio for a venous thromboembolic disease associated with circulating titers of anti-C. pneumoniae IgG antibodies of 256 or more was 6.2 (95% CI; 3.8-10.1) and remained high after exclusion of the patients with an Arg 506 Gln mutation in factor V and a G 20210 A mutation in factor II (odds ratio 7.7; 95% CI; 4.5-13.2). The fact that the odds ratio for a venous thromboembolic disease increases with the IgG antibody titer reinforces this association (cf. table 3).

TABLE 1
Characteristics of the cases with a venous
thromboembolic disease (TED) and of the controls.

| | Cases | Controls | |
|---|---|---|---|
| | n = 176 | n = 197 | Test |
| % women | 56.2 | 55.3 | p = 0.87 |
| Mean age (SD) | 42.8 (10.7) | 42.9 (10.6) | p = 0.92 |
| % oral contraception in | 34.0 | 22.0 | p = 0.053 |
| women | | | |
| % FV mutation-Arg 506Gln | 21.9 | 5.1 | p < 0.0001 |
| % FII mutation-G20210A | 10.2 | 4.1 | p = 0.02 |
| % spontaneous TED | 37.6 | — | — |
| % recurrent TED | 26.7 | — | — |
| % pulmonary embolism | 37.1 | — | — |
| Mean age at first TED | 38.4 (12.0) | — | — |
| (SD) | | | |

TED: venous thromboembolic disease
SD: standard deviation

TABLE 2
Detection and titration of anti-Chlamydia pneumoniae IgG antibodies in
the cases and controls, and odds ratios (95% CI) for a venous
thromboembolic disease (TED) associated with IgG titers ≧ 256

| C. pneumoniae | Controls | All cases | | Cases < 3 months* | |
|---|---|---|---|---|---|
| IgG titers | n(%) | n(%) | OR(95% CI) | n(%) | OR(95% CI) |
| Negative | 57(29.2) | 44(25.0) | | 26(29.9) | |
| 128 | 107(54.9) | 37(21.0) | | 17(19.5) | |
| 256 | 17(8.7) | 27(15.4) | | 14(16.1) | |
| | | | 6.2(3.8-10.1)** | | 5.4(3.1-9.6)** |
| 512 | 13(6.7) | 43(24.4) | p < 0.0001 | 20(23.0) | p < 0.0001 |
| 1024 | 1(0.5) | 25(14.2) | | 10(11.5) | |

*Cases with blood samples taken within three months after the TED episode.
**<256 vs ≧256
CI: confidence interval
OR: odds ratio

TABLE 3
Odds ratios (95% CI) for a venous thromboembolic disease (TED) as a
function of the anti-Chlamydia pneumoniae IgG titers with reference
to the absence of detectable antibodies.

| C. pneumoniae | All cases | | | Cases < 3 months* | | |
|---|---|---|---|---|---|---|
| IgG titers | OR | (95% CI) | p | OR | (95% CI) | p |
| Negative | 1 | — | — | 1 | — | — |
| 128 | 0.4 | (0.3-0.8) | 0.004 | 0.3 | (0.2-0.7) | 0.0028 |
| 256 | 2.1 | (1.0-4.2) | 0.05 | 1.8 | (0.8-4.2) | 0.17 |
| 512 | 4.3 | (2.1-8.9) | <0.0001 | 3.4 | (1.5-7.8) | 0.0045 |
| 1024 | 32.4 | (4.2-248.3) | 0.0008 | 21.9 | (2.7-180.3) | 0.0041 |

*Cases with blood samples taken within three months after the TED episode
CI: confidence interval
OR: odds ratio

### References

Bartels C. et al. Detection of Chlamydia pneumoniae but not cytomegalovirus in occluded saphenous vein coronary artery bypass grafts. Circulation, 1999, 99:879-882.

Gibbs RGJ, Carey N, Davies AH. Chlamydia pneumoniae and vascular disease. Br J Surg 1998;

85:1191-7.

Ong G, Thomas BJ, Mansfield AO, Davidson BR, Taylor-Robinson D. Detection and widespread distribution of Chlamydia pneumoniae in the vascular system and its possible implications. J. Clin Pathol 1996; 49:102-6.

Rosendaal FR. Thrombosis in the young: epidemiology and risk factors. A focus on venous thrombosis. Thromb Haemost 1997; 78:1-6.

1. A method for preventing and/or treating thromboembolic disease wherein a phrophylactically or therapeutically effective amount of an agent active on bacteria of the Chlamydia genus, in combination with a pharmaceutically acceptable carrier, is administered to a patient requiring such treatment.

2. The method of claim 1, wherein the Chlamydia is Chlamydia pneumoniae.

3. A method to claim 1, wherein said active agent is an antibiotic agent.

4. The method of claim 3, wherein the antibiotic agent is selected from the group consisting of macrolides, tetracyclines, fluroquinolones and rifampicin.

5. The method according to claim 1 intended for the prevention of recurrences subsequent to a first venous thrombosis.
